(19) United States
(12) Patent Application Publication
(10) Pub. No.: US 2013/0324567 A1
(43) Pub. Date: Dec. 5, 2013

(54) RESTORATION OF HAIR

(71) Applicant: Shaiw-Wen Liu, Honeybrook, PA (US)

(72) Inventor: Shaiw-Wen Liu, Honeybrook, PA (US)

(73) Assignee: Conrex Pharmaceutical Corporation, Newtown Square, PA (US)

(21) Appl. No.: 13/825,482

(22) PCT Filed: Sep. 21, 2011
(86) PCT No.: PCT/US2011/052576

§ 371 Date: Aug. 5, 2013

Related U.S. Application Data
(60) Provisional application No. 61/384,825, filed on Sep. 21, 2010.

(51) Int. Cl. A61K 8/49; A61Q 7/00

(52) U.S. Cl. CPC A61K 8/4953; A61Q 7/00
USPC 514/275

(57) ABSTRACT

A nonirritant composition which is useful in restoring hair and which comprises a normally solid, water-insoluble hair restorer, for example, minoxidil, a normally solid Hsieh dermal penetration-facilitator for the hair restorer, for example, pentadecalactone, and a solvent or co-solvent in which the hair restorer and the facilitator are solubilized and which does not impart to the composition irritant properties, and wherein, upon application to the surface of the skin of a recipient, the hair restorer penetrates the dermal layer of the skin and with minimal systemic delivery of the hair restorer into the blood stream of the recipient.

### CROSS REFERENCE TO RELATED APPLICATION(S)

This application is the National Stage filing of PCT Application No. PCT/US2011/052576, filed Sep. 21, 2011, which claims the benefit of U.S. Provisional Application 61/384,825, filed Sep. 21, 2010, the entire disclosure of which is hereby incorporated by reference.

### FIELD OF THE INVENTION

The present invention relates to a composition which upon application to the surface of the skin is effective in restoring hair growth for the benefit of the recipient, for example, an individual with alopecia (baldness). As disclosed in variously issued patents, for example, as identified hereinbelow, it is known to treat the skin of an individual who is suffering or has suffered hair loss, typically on the scalp of the individual, with one or more compounds which are hair restorers. Such compounds are effective, for example, in stimulating the change of vellus hair (fine colorless hair whose presence is hard to see) to terminal hair. Vellus hair is a precursor of terminal hair which has a relatively large diameter, is colored, and whose presence is readily observable; in addition, such compounds can be effective in increasing the rate of growth of terminal hair.

### BRIEF DESCRIPTION OF THE INVENTION

Popularly used hair restorers are typically solids and have poor water solubility. For effective application to the skin, the water-insoluble, solid restorers are solubilized by use of organic solvents. Unfortunately, popularly used organic solvents impart to the composition comprising the solubilized hair restorer properties which irritate the skin of the recipient.

Broadly stated, the present invention provides a nonirritant composition comprising a solubilized form of a hair restorer, a dermal penetration-facilitator for the hair restorer, and a solvent which is effective in solubilizing the hair restorer and the facilitator and which does not impart to the composition properties which irritate the skin of the recipient.

In preferred form, the composition comprises: (A) an “iminopyrimidine” hair restorer as described in detail below, most preferably minoxidil; (B) a Hsieh enhancer dermal penetration-facilitator, most preferably pentadecalactone; and (C) a non-irritant co-solvent. An optional, but preferred ingredient, is an oxygen carrier.

### DETAILED DESCRIPTION OF THE INVENTION

The composition of the present invention contains a hair restorer, that is, a compound that slows hair loss or stops hair loss or promotes hair growth or re-growth; the hair restorer may function in more than one way of the aforementioned ways. Accordingly, the composition can be used to treat, for example, androgenetic alopecia.

U.S. Pat. No. 3,461,461 discloses a class of compounds which have various uses including, for example, as antihypertensive agents and anti-inflammatory agents; this patent discloses also a method for preparation of the compounds. U.S. Pat. No. 4,139,619 discloses that the class of compounds disclosed in the '461 patent are capable of functioning as a hair restorer. Compounds disclosed in the aforementioned patents, including a mixture of two or more of the compounds, can be used as a hair restorer in the composition of the present invention. The disclosure of each of the aforementioned patents is incorporated herein by reference. For convenience, the aforementioned compounds are referred to also herein as “pyrimidine”.

Compounds disclosed in the aforementioned '461 and '619 patents are encompassed within the formula: 6-amino-1,2-dihydro-1-hydroxy-2-iminopyrimidines and include their carboxyacylated counterparts and the corresponding acid addition salts; they are substituted in the 4-position and optionally in the 5-position, the substituent in the 4-position being a secondary or tertiary amino moiety.

The preferred compound for use as a hair restorer in the practice of the present invention is minoxidil; the use of minoxidil as a hair restorer is disclosed and claimed particularly in U.S. Pat. No. 4,596,812, the disclosure of which is incorporated herein by reference also. Minoxidil is known also, for example, as: 2,3-dihydro-3-hydroxy-2imino-6-(1-piperidinyl)-4-pyrimidinamine; and 2,4-diamino-6-piperidino-pyrimidin3-oxide. At ambient conditions, minoxidil is a crystalline powder which is slightly soluble in water, but soluble in ethanol and propylene glycol. For use in the practice of the present invention, the hair restorer is solubilized, as described hereinafter

The hair restorer should be present in the composition in an amount which is effective to restore hair to the desired extent. Such amount will vary depending on various factors, for example: the particular compound being used; the nature of the condition being treated, for example, the severity of the hair loss; the individual being treated, that is, male or female; and the nature of other ingredients comprising the composition. It is believed that, for most applications, the amount of the hair restorer (or a mixture of two or more compounds comprising the hair restorer) will comprise about 2 to about 20 wt. % of the composition. (Unless stated otherwise, the term “wt. %” when used herein means weight % based on the total weight of the composition.) In the treatment of the skin of a male, it is preferred that the composition comprise about 2 to about 15 wt. % of the hair restorer and in the treatment of the skin of a female, it is recommended that the composition comprise about 2 to about 5 wt. % of the composition.

The dermal penetration-facilitator (also referred to herein for convenience as “the facilitator”) of the composition of the present invention functions to promote the delivery of the hair restorer to the dermal layer of the skin and to the hair follicle and/or hair bulb, with minimal or no systemic delivery of the facilitator into the blood stream of the individual. While not wishing to be bound by any specific theory, it is believed that the facilitator causes an increase in the fluidity of the overlapping cell membranes of the stratum corneum (outermost surface layer of the skin), thereby facilitating diffusion of the hair restorer through the cells as well as around them. Thus, the facilitator acts like a solvent to enhance the solubility of the hair restorer in the epidermal and follicular cell membrane lipids. The permeation-enhancing effect of the facilitator is predictable, temporary, and does not cause irritation or any long term change to the skin structure. A mixture of two or more of the facilitators can be used.

The preferred facilitator for use in the composition of the present invention is a member of the class of compounds which have become known in the art as “Hsieh enhancers”, for example, as described in U.S. Pat. No. 5,023,252 to Hsieh. This patent describes, however, that the Hsieh enhancers function as transdermal- or transmembrane-delivering compounds that enhance the delivery of the drug from the surface of the skin or nasal membrane into the blood stream of the individual. Accordingly, in accordance with the disclosure of the Hsieh patent, the Hsieh enhancers are used in applications in which they are combined with a drug, which to be effective, must be delivered systemically into the blood stream of the recipient. Examples of drugs which can be delivered systemically by such enhancers include the peptide insulin and the steroid fluorogestorone acetate.

In the present invention, the Hsieh enhancer is used in a different way, that is, as a penetration-facilitator for delivery of the hair restorer to the dermal layer of the skin and hair follicle without significant, if any, systemic absorption of the hair restorer into the blood stream of the user. In order to accomplish this, the concentration of the dermal penetration-facilitator is adjusted, typically lowered, in a manner dependent on the specific hair restorer used, in order to promote delivery of an effective amount of the hair restorer through the epidermis and to the dermal layer and hair follicles of the skin, but not to the bloodstream.

Examples of Hsieh enhancers that can be used in the composition are 3-methylcyclopentadecanone (muscone), 9-cycloheptadecen-1-one (civetone), cyclohexadecanone, and cyclopentadecanone (normuscone). As mentioned above, the use of pentadecalactone is preferred.

It is believed that, for most applications, the effective amount of the facilitator of the present invention will fall in the range of about 1 to about 10 wt. %, depending on the specific hair restorer to be delivered and the condition to be treated. The preferred amount of the penetration-facilitator is be about 2 to about 5 wt. %.

Solid forms of the hair restorer and facilitator should be present in the composition of the present invention in solubilized form. As mentioned above, the composition can be formulated as a nonirritant composition which distinguishes it from prior art compositions that have a tendency to irritate the scalp. The term “nonirritant composition” means that, upon application of the composition to the outer surface of skin (epidermis) comprising the scalp, the composition does not cause the recipient thereof to experience discomfort to a degree such that the recipient tends to want to refrain from use of the composition as prescribed. The discomfort can manifest itself in various ways, for example, redness, drying and itching, and excessive drying of hair itself making it less flexible.

In prior art “irritating” compositions, it is typically the solvent(s) which is/are responsible for imparting this undesirable property to the composition. The hair restorer, for example, minoxidil, is highly insoluble or indeed insoluble in water. Organic solvents which are effective in solubilizing the hair restorer may be inappropriate for use because they are considered toxic for human applications; those which are non-toxic may have, nevertheless, other undesirable characteristics, for example, functioning as an irritant when applied to the skin or causing the dissolved hair restorer to be delivered directly into the blood stream of the individual.

In accordance with the present invention, the solvent or co-solvent is such that it is effective in dissolving the normally water-insoluble ingredients of the composition and it is a “nonirritant”, that is, the composition containing it is a nonirritant composition. There are solvents which are effective in dissolving the insoluble ingredients, but to be effective solubilizers, they have to be used in amounts such that they impart to the composition irritating properties. In accordance with the present invention, such solvents can be used, but in amounts which are “nonirritating” and which do not dissolve satisfactorily the insoluble ingredients, provided that the composition includes another solvent in an amount which is “non-irritating” and, nevertheless, an amount which with its co-solvent is effective to solubilize satisfactorily the insoluble ingredients.

By way of example, it is noted that ethanol is a popularly used solvent in known hair-restorer compositions because it has excellent solubilizing properties; however, in the amounts used typically, it is an irritant. In preferred form the composition of the present invention is “ethanol-free” or if ethanol is used with a co-solvent, it is used in an amount which does not impart irritating properties to the composition.

In preferred form, the co-solvents are miscible with each other, each solvent is miscible with the dissolved solids and is water-miscible, and has antimicrobial properties, and aids also as a facilitator for promoting delivery of the hair restorer through the skin.

In preferred form, the composition comprises a co-solvent consisting essentially of pentylene glycol (solvent A) and propylene glycol (solvent B).

The solvent or co-solvent is present in the composition in an amount sufficient to dissolve both the hair restorer and the facilitator. The amount will depend on various factors including, for example, the particular solvents being used and the particular hair restorer and facilitator being used and also the amounts thereof. For most applications, it is believe that the amount of solvent or co-solvent comprising the composition will be about 1 to about 40 wt. %, with the ratio of solvent A to solvent B being in the range of about 1:1 to about 0.1:20.

In a preferred embodiment, the composition of the present invention includes also a liquid organic compound which is capable of dissolving oxygen and which, accordingly, is an oxygen carrier, for example, capable of having dissolved therein, for example, at least about 40 ml of oxygen per 100 ml of the oxygen carrier at 25° C.; such compounds are known and are referred to hereafter as an “oxygen carrier”. A preferred class of oxygen carriers for use in the present invention are perfluorocarbons. A preferred oxygen carrier for use in the present invention is perfluorodecalin which is basically decalin, that is, decahydronaphthalene, in which all of the hydrogen atoms have been replaced by fluoride atoms. At ambient conditions, perfluorodecalin is a liquid which is non-toxic, non-volatile, odorless, colorless, and insoluble in water.

The oxygen carrier should be present in the composition in an amount at least sufficient to supply supplementary oxygen to the hair follicle and/or hair bulb. The amount may vary depending on various factors, for example, the particular constituents comprising the composition and the amounts thereof and the particular form of the composition. For most applications, it is believe that an amount comprising about 1 to about 30 wt. % of the composition will be satisfactory.

The composition of the present invention can be in any form which is suitable for application to the skin of a recipient for appropriate transfer of the hair restorer through the skin. Examples of such forms include liquids of varying viscosities, including a viscosity that enables the composition to be sprayed readily onto the surface of the scalp, and thickened compositions which can be applied topically to the surface of the scalp; in such thickened form, the composition should have a viscosity that enables it to be spread readily onto the surface of the scalp and such that it has sufficient cohesiveness to retain substantially its shape and presence after application thereto. In addition, the composition can be formulated so that it can be applied by use of a transdermal skin patch which contains the composition.

In formulating a thickened form of the composition, any compound(s) which does not impart to the composition irritant properties and which is capable of thickening the dissolved drug and facilitator to form a thickened composition as described above can be used. Typically, such compounds comprise thickening agents; a mixture of thickening agents can be used. Preferred thickening agents for use in the composition of the present invention are alkylacrylates.

The amount of thickening agent comprising the composition will depend on various factors, including, for example, the nature of the other ingredients of the composition and the amounts thereof and also the particular form in which it is desired to use the composition. The non-thickened composition comprises the solid drug and facilitator dissolved in organic solvent(s) and is typically in the form of a non-shape-retaining runny liquid. The thickener is used in an amount that converts the liquid into the particular form in which it is desired to use the composition. It is believed that, for most applications, this can be accomplished by the use of about 5 to 20 wt. % of the thickening agent or mixture of thickening agents.

Optionally, the composition can include also one or more other materials which impart to the composition desired properties and which do not impart irritant properties to the composition. Such materials will comprise typically compounds of the type that are used as additives in pharmaceutical compositions. Examples of such materials are preservatives, pH controlling agents, stabilizers, surfactants, and emulsifiers. It should be understood that the composition of the present invention should be free of any material that would cause the composition to be irritating in use. Such optional materials are used typically in an amount comprising about 10 to about 20 wt. % of the composition.

Additional optional ingredients for use in the composition are compounds which function as hair restorers, but are different from the “pyrimidine” hair restorers described hereinabove. Examples of such compounds are those known as nonspecific promoters of hair growth, for example, tretinoin, nicotinate, and azelair acid. Another exemplary group of hair restorers is known as 5-redictase inhibitors, for example, finasteride, dutasteride, and saw palmetto. In the use of such optional hair restorers, they will generally comprise about 1 to about 20 wt. % of the composition and should comprise preferably no more than about 20 wt. % of the total amount of hair restorer comprising the composition.

The composition can be applied to the skin as often as needed to achieve the desired results, that is, to achieve hair growth stimulation. The frequency of application can vary depending on various factors, for example, the nature of the composition and the involved condition. In general, application of the composition one or two times a day will be suitable and can be continued for as long as required to obtain the desire results, for example, weeks or months or indefinitely. It is believed that application of the composition to the skin on a daily basis in an amount such that about 0.001 g to about 10 g of the hair restorer is applied to the skin will be sufficient to obtain the desired results.

### EXAMPLES

The following examples are illustrative of nonirritant compositions of the present invention and the use thereof.

### Example No. 1

This example is illustrative of a topical gel which contains minoxidil hair restorer and the dermal penetration-facilitator pentadecalactone.

| Ingredient | Amount, wt. % |
|---|---|
| minoxidil | 5 |
| pentadecalactone | 4 |
| pentylene glycol (solvent) | 38 |
| propylene glycol (solvent) | 37 |
| acrylates/C₁₀₋₃₀ | 15 |
| alkyl acrylate crosspolymer | |
| (thickening agent) | |
| glycerol caprylate & | 1 |
| methylisothiazolinone | |
| (stabilizing agent) | |

A composition comprising the above constituents can be prepared in any suitable way to form a gel. For example, the composition can be prepared by mixing the liquid co-solvents propylene glycol and pentylene glycol together in a container. With constant stirring, add the solid minoxidil into the co-solvent solution which is maintained, as it is heated, at 50° C. in a water bath to melt all of the minoxidil. With stirring, add the pentadecalactone to the contents of the container as it is maintained in the water bath. With stirring, add the thickening agent to the contents of the container as it is maintained in the water bath. And with stirring, add the stabilizing and preservative agents to the contents of the container as it is maintained in the water bath at a temperature of 35° C. The resulting gel can be spread onto the surface of the skin of the scalp of a human male or female for delivery of the minoxidil to the hair follicle.

It is recommended that there be applied once daily to the skin about 1 to about 5 mls of the composition and that such application be continued daily for a period of time of about 3 to about 6 months.

### Example No. 2

This example is illustrative of a liquid composition containing minoxidil, the dermal penetration-facilitator pentadecalactone, and the oxygen carrier perfluorodecalin.

| Ingredient | Amount, wt. % |
|---|---|
| minoxidil | 5 |
| pentadecalactone | 4 |
| pentylene glycol (solvent) | 35 |
| propylene glycol (solvent) | 35 |
| perfluorodecalin | 20 |
| (oxygen carrier) | |
| glycerol caprylate & | 1 |
| methylisothiazolinone | |
| (stabilizing agent) | |

A composition comprising the above constituents can be prepared in any suitable way to form a liquid solution of the ingredients. For example, the composition can be prepared in the same manner used to prepare the composition of Examiner No. 1, except that the oxygen carrier, instead of the thickening agent, is added to the contents of the container. The resulting composition is a clear liquid solution that is oily in nature; it can be applied by spraying onto the surface of the skin of the scalp of a human male or female for delivery of the minoxidil to the hair follicle. It is recommended that there be applied once daily to the scalp about 1 to 5 ml of the composition and that such application be continued daily for a period of time of about 3 to about 6 months.

Two additional embodiments of compositions within the scope of the present invention are compositions like those of Example Nos. 1 and 2 except modified by replacing the stabilizing agent thereof with a mixture of diazolidinyl urea, methylparaben, and propylparaben.

1. A nonirritant composition which is useful in restoring hair and which comprises a normally solid, water-insoluble hair restorer, a normally solid Hsieh dermal penetration-facilitator for the hair restorer, and a solvent or co-solvent in which the hair restorer and the facilitator are solubilized and which does not impart to the composition irritant properties, and wherein, upon application to the surface of the skin of a recipient, the hair restorer penetrates the dermal layer of the skin and with minimal systemic delivery of the hair restorer into the blood stream of the recipient.

2. A composition according to claim 1 which includes also an oxygen carrier.

3.-11. (canceled)

12. A nonirritant composition which is useful in restoring hair and which comprises (A) about 2 to about 20 wt. % of a normally solid, water-insoluble hair restorer; (B) about 1 to about 10 wt. % of a normally solid Hsieh dermal penetration-facilitator for the hair restorer; and (C) and a solvent or co-solvent in which the hair restorer and the facilitator are solubilized and which does not impart to the composition irritant properties; optionally, about 1 to about 30 wt. % of an oxygen carrier; and (E) optionally, about 10 to about 20 wt. % of at least one of the following: a preservative or stabilizing agent, a pH controlling agent, and a surfactant; and (F) optionally, about 5 to about 20 wt. % of a thickening agent and wherein, upon application to the surface of the skin of a recipient, the hair restorer penetrates the dermal layer of the skin and with minimal systemic delivery of the hair restorer into the blood stream of the recipient.

13. In a method for treating the surface of the skin of a patient to restore hair growth, the improvement comprising topically treating the skin with a composition according to claim 12.

14. A composition according to claim 1 which includes minoxidil hair restorer.

15. A composition according to claim 14 which comprises: about 2 to about 15 wt. % of said minoxidil hair restorer; about 2 to about 5 wt. % of said penetration-facilitator; and about 1 to about 40 wt. % of said cosolvent consisting essentially of pentylene glycol and propylene glycol.

16. A composition according to claim 12 in which said penetration-facilitator is pentadelclactone.

17. A composition according to claim 15 in which said penetration-facilitator is pentadecalactone.

18. A composition according to claim 12 including pentylene glycol solvent in an amount of about 1 to about 40 wt. %.

19. A composition according to claim 15, wherein the weight ratio of pentylene glycol to propylene glycol is within the ration of about 1:1 to about 0.1:20.

20. A composition according to claim 12 including a fluorocarbon oxygen carrier.

21. A composition according to claim 20 in which the oxygen carrier is perfluorodecalin.

22. A composition according to claim 15 including about 1 to about 30 wt. % of a fluorocarbon oxygen carrier.

23. A composition according to claim 22 wherein the oxygen carrier is perfluorodecalin.

24. A composition according to claim 12 wherein said penetration-facilitator is selected from the group consisting of 3-methylcyclopentadecanone (muscone), 9-cycloheptadecen-1-one (civetone), cyclohexadecanone, and cyclopentadecanone (normuscone).

25. A composition according to claim 14 including said optional ingredients, namely (D), (E) and (F).

26. A composition according to claim 15 including said optional ingredients, namely (D), (E) and (F).

27. A method according to claim 13 wherein the scalp is treated with said composition.

28. A method according to claim 27 wherein said composition includes minoxidil hair restorer.

29. A method according to claim 27 wherein the composition is applied to the scalp on a daily basis in an amount of about 0.001 to about 10 grams.
